(19) United States
(12) Patent Application Publication
(10) Pub. No.: US 2019/0376124 A1
(43) Pub. Date: Dec. 12, 2019

(54) APPARATUS AND METHOD FOR SPECIFIC DETECTION AND QUANTIZATION OF NUCLEIC ACID

(71) Applicant: Takena Technologies, Inc., Albany, OR (US)

(72) Inventors: David Clayton Roberts, Albany, OR (US); Gregory Allen Peek, Forest Grove, OR (US); John Enrique Mata, Corvalis, OR (US)

(73) Assignee: Takena Technologies, Inc., Albany, OR (US)

(21) Appl. No.: 16/306,314

(22) PCT Filed: Jun. 1, 2017
(86) PCT No.: PCT/US17/35515

§ 371 Date: Nov. 30, 2018

Related U.S. Application Data
(60) Provisional application No. 62/344,881, filed on Jun. 2, 2016.

(51) Int. Cl. C12Q 1/6818; C12Q 1/70; G01N 21/64

(52) U.S. Cl. CPC C12Q 1/6818; C12Q 1/701; G01N 2021/6439; C12Q 2600/112; G01N 21/6428

(57) ABSTRACT

A method for detecting and quantifying a target nucleic acid sequence comprises combining a first phosphorodiamidate morpholino oligomer (PMO) probe with a donor dye, the first probe having a sequence complementary to a first portion of the of the target nucleic acid sequence, a second PMO probe comprising a receptor dye, the second probe having a sequence complementary to a second portion of the target nucleic acid sequence, in near proximity to the first portion; and a sample material, exposing the combined solution to incident light to cause the first fluorescent dye to donate energy to the second fluorescent, causing the second fluorescent dye to fluoresce, and detecting and measuring fluorescence of the second fluorescent dye.

### CROSS-REFERENCE TO RELATED APPLICATIONS

The present invention claims priority to a provisional patent application 62/334,881, filed on Jun. 2, 2016, and all disclosure of the parent case is incorporated herein at least by reference.

This application contains a Sequence Listing text file submitted via EFS Web on Aug. 22, 2019, the entire contents of which are hereby incorporated by reference. The text file has a file name, dnaProfileST25.txt, created on Apr. 16, 2019 and a file size of 1093 Bytes.

### BACKGROUND OF THE INVENTION

### 1. Field of the Invention

The present invention is in the technical field of diagnostics, and pertains more particularly to detecting and quantifying specific nucleic acids, particularly viruses.

### 2. Description of Related Art

Conventional methods for detection of nucleic acids typically require enzymatic amplification of samples, and molecular methods that are difficult to replicate outside of a laboratory environment. Such conventional methods thus are not usable in field environments, where temperatures may not be precisely controllable, and trained and experienced personnel may not be available. What is therefore clearly needed is a method and apparatus for such detection and quantification that is relatively

uncomplicated and efficient, and may be performed by relatively inexperienced persons.

### BRIEF SUMMARY OF THE INVENTION

In one embodiment of the present invention a method for detecting and quantifying a target nucleic acid sequence in a sample is provided, comprising the steps of combining in a single solution a first phosphorodiamidate morpholino oligomer (PMO) probe comprising a first fluorescent dye actionable under exposure to incident light to act as a donor, the first probe having a sequence complementary to a first portion of the of the target nucleic acid sequence, a second PMO probe comprising a second fluorescent dye actionable as a receptor, the second probe having a sequence

complementary to a second portion of the target nucleic acid sequence, the second portion in near proximity to the first portion, and a sample material suspected of comprising strands of the target nucleic acid sequence, exposing the combined solution to incident light at a specific wavelength known to cause the first fluorescent dye of the first PMO probe to donate energy in a non-radiative manner to the second fluorescent dye of

the second PMO probe, causing the second fluorescent dye to fluoresce as a result, and detecting and measuring fluorescence of the second fluorescent dye by a light detection device, detection indicating presence of the target nucleic acid sequence in the sample.

In one embodiment, the first and second PMO probes are added together or sequentially to a solution containing the sample material suspected of comprising strands of the target nucleic acid sequence. Also in one embodiment, the sample material suspected of comprising strands of the target nucleic acid sequence is added to a solution containing the first and second PMO probes. In one embodiment, the light detection device is enabled to record intensity of the emitted photons as an indication of the quantity of the target nucleic acid present. And in one embodiment the method further

comprises adding a quencher dye having substantially lower binding affinity than the target nucleic acid, which quencher dye binds to the acceptor dye in solution, absorbing energy from the acceptor dye when the second PMO probe is free in solution, and disassociates when and if the second PMO probe binds to the target nucleotide.

In one embodiment, the first and second PMO probes contain multiple donor or acceptor dyes, enabling multiplexed amplification of signal from more than two oligomers. Also in one embodiment, the first and second PMO probes are prepared complementary to one of the nucleic acid sequences Ebola virus, Zika virus, tuberculosis virus, salmonella virus, HIV virus, or HCV virus. Also in one embodiment, the method further comprises a step for comparing result of measurement of light emission of the second fluorescent dye to prepared standards, to determine quantity of target nucleic acid sequence in the sample.

In another aspect of the invention a kit for detecting and quantifying a target nucleic acid sequence in a test sample is provided, comprising a first container holding a sample solution possibly comprising the target nucleic acid, a second container holding, in solution, a first phosphorodiamidate morpholino oligomer (PMO) probe comprising a first fluorescent dye actionable under exposure to incident light to act as a donor, the first PMO probe having a sequence complementary to a first portion of the of the target nucleic acid sequence, a third container holding, in solution, a second PMO probe comprising a second fluorescent dye actionable as a receptor, the second probe having a sequence complementary to a second portion of the target nucleic acid sequence, the second portion in near proximity to the first portion, a light source enabled to illuminate contents of the first container at a wavelength inducing the first fluorescent dye to donate energy in a non-radiative manner to the second fluorescent dye, and a light-detection device to collect and measure fluorescent light emissions from the acceptor probe dye.

In one embodiment, the kit further comprises a fourth container holding, in solution, a quencher dye having substantially lower binding affinity than the target nucleic acid, which binds to the acceptor dye in solution, absorbing energy from the acceptor dye when the second oligomer probe is free in solution, and disassociates when and if the second oligomer probe binds to the target nucleotide. Also in one embodiment, the light detection device is enabled to record intensity of the emitted photons as an indication of the quantity of the target nucleic acid present. In one embodiment, the first and second PMO probes contain multiple donor or acceptor dyes, enabling multiplexed amplification of signal from more than two probes. Also in one embodiment, the first and second oligomer probes in the second and third containers, respectively, are prepared to be complementary to one of the nucleic acid sequences Ebola virus, Zika virus, tuberculosis virus, salmonella virus, HIV virus, or HCV virus.

In yet another aspect of the invention a portable detection system for detecting and quantifying a target nucleic acid sequence in a sample is provided, comprising a hand-held, computerized appliance having a cartridge holder for holding a sample cartridge, a laser for illuminating a sample in a cartridge in the cartridge holder, a light sensor for detecting light emission from the sample in the cartridge, and wireless circuitry for communication with a network-connected communication device, and a cartridge configured to insert and withdraw from the cartridge holder, the cartridge comprising a donor probe chamber, an acceptor probe chamber, a sample chamber, and a target reaction chamber, with interconnecting internal channels. A cartridge implemented with donor and acceptor probes specific to a particular target nucleotide in respective chambers is loaded with a sample in the sample chamber, and inserted into the cartridge holder, where the sample and probes are mixed in the reaction chamber and illuminated by the laser, and intensity determined by the light detector is communicated wirelessly to the network-connected communication device.

In one embodiment, the portable detection system further comprises an application executing on the network-connected communication device, enabling digital storage of results of assays and communication with a remote, network-connected database. Also in one embodiment, the system further comprises the cartridge having machine readable information thereon, identifying at least a unique serial number for tracking, and further comprising apparatus in the hand-held, computerized appliance, enabling reading of the machine-readable indicia. Also in one embodiment, the machine-readable information is in the nature of a bar code or Quick Response (QR) code, and the reading apparatus is optical in nature. In one embodiment, the machine-readable indicia is electronic in nature, and the reading apparatus comprises wireless circuitry for accessing the data on the cartridge. In one embodiment, the reading apparatus comprises electronic contacts between the cartridge and circuitry in the hand-held device. And in one embodiment, in addition to a unique serial number, the information comprises one or more of an assay name, date of manufacture and lot number, and one or more data tables.

### BRIEF DESCRIPTION OF THE SEVERAL VIEWS OF THE DRAWINGS

FIG. 1A is a schematic representation of a single strand of a nucleotide in a sample solution ostensibly containing a quantity of the subject nucleotide.

FIG. 1B is a schematic representation of the nucleotide strand of FIG. 1A with a donor probe oligomer comprising a fluorescent substance, hybridized at a first specific location, and further probes in a step in a process for identifying quantifying the nucleotide.

FIG. 1C is a schematic representation of the nucleotide strand of FIGS. 1A and 1B, and the donor probe, having a second emitter probe hybridized to the nucleotide strand at a second specific location proximal to the specific location of the donor probe, including other elements and process in an embodiment of the present invention.

FIG. 2 is a graph illustrating a time course of 1 nM Donor DNA Probe compared to a Morpholino Probe binding to 1 nM target DNA.

FIG. 3 illustrates DNA probes labeled with Alexa488 and Alexa 546 bound to target DNA.

FIG. 4 illustrates Morpholino probes labeled with FITC and Alexa546 bound to target DNA.

FIG. 5 illustrates a generalized kit in an embodiment of the invention.

FIG. 6 illustrates a diagnostic device in another embodiment of the invention. FIG. 7 is a plan view of one diagnostic assay cartridge in an embodiment of the invention.

FIG. 7 is a plan view of one diagnostic assay cartridge in an embodiment of the invention.

FIG. 8 is a block diagram of the elements of diagnostic device in an embodiment of the invention.

FIG. 9 is a flow chart illustrating a process cycle in one embodiment of the invention.

FIG. 10 illustrates a simple, serial process that is performed for each nucleotide that is expected to be a target for analysis by the system of the invention.

### DETAILED DESCRIPTION OF THE INVENTION

The invention in one embodiment relates to apparatus and methods for rapidly detecting a target nucleic acid sequence from a complex matrix. In this embodiment, a sample is exposed to a stabilizing and denaturing reagent, followed by rapid purification and introduction of target-specific probes. When bound in proper conformation and illuminated by an appropriate wavelength of light, a fluorescent substance attached to a donor probe will transfer energy suitable to activate a fluorescent substance attached the acceptor probe. Light emitted from the acceptor probe will then be collected and quantified for determination of presence and enumeration of molecules present that are specific matches for the target nucleic acid sequence. This method of detection is suitable for benchtop apparatus or for small portable devices. The invention in some embodiments includes a kit for the detection of nucleic acids, the kit comprising reagents prepared to be specific to a target nucleic acid sequence, such as, for example, found exclusively in a particular virus, manipulation containers for sample preparation and mixing with reagents, and a detector for receiving and quantifying light emission from treated samples.

There are numerous circumstances in the real world, particularly, but not exclusively to developing cultures, and third-world countries, wherein the apparatus and method of the invention will provide a huge advantage over conventional apparatus and methods, and make detection and tracking of disease organisms a much more achievable and exact science. As a simple example, the emergence and spread of the Ebola virus in West Africa in recent memory, was a serious danger to millions of citizens of the countries affected, and raised an urgent need to test and detect persons moving from place-to-place, and country-to-country, to try to contain the outbreak. The equipment, personnel and cost for this effort was quite substantial. With the methods and apparatus of the present invention, the tracking and containment could have been accomplished very much quicker, and at a far less cost than was the case with conventional practice.

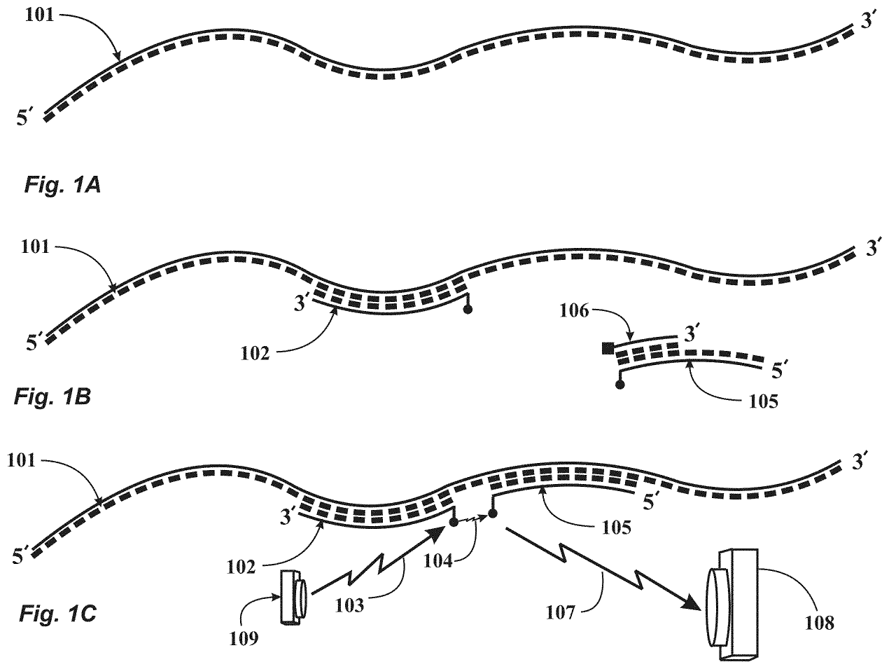

FIGS. 1A, 1B and 1C are consecutive schematic representations of steps in a process of nucleotide, particularly pathogen, detection in one embodiment of the present knowledge of nucleotide sequence of various pathogens for which it is desirable to have a low-cost and rapid means of detection. These nucleotide sequences for such pathogens is fortunately very well known. Further, methods to purify and concentrate nucleic acids are common and well known to those skilled in the art. Consequently the process and steps for collecting a sample, and concentrating and purifying same, are not described herein in detail.

At a substantially abstract level, the inventors surmised that they could synthesize oligonucleotide probes that would be complementary to portions of the nucleotide sequence of a target pathogen, allowing the probes to be hybridized to the sequence of the target nucleotide. Taking a single pathogen as an example, and supposing that a sample has been prepared, purified and suitably concentrated, and the supposed pathogen in the sample is unknown, if the probes prepared are specific to a suspected pathogen, Ebola, for example, and the probes, after being added to the sample, do not hybridize to the nucleotide of the unknown sample, it is reasonable to conclude that the sample is not, in fact, the suspected pathogen. If, however, the probes do hybridize to the sample, then it is reasonable to conclude that the suspected pathogen is indeed present in the sample. So, the question becomes, how to determine if the binding took effect, or not.

In one embodiment of the invention, FIG. 1A represents a single strand nucleotide 101 in the prepared sample, having 5′ and 3′ ends. There will, of course, be a substantially greater plurality of the target nucleotide in the sample. Two probes designed to be complementary in sequence to the sequence of the suspected pathogen, are added to the sample, or, in some embodiments, the sample is added to a solution containing the two probes. A first of the probes is illustrated as probe 102 in FIG. 1B. Probe 102 is designed as, and designated a donor probe, because in its design at least one fluorescent substance is conjugated to probe 102, which substance will act as a donor. A second probe 105 is designed as a receptor fluorophore, having a sequence to be complementary to the sequence of the target at a different location than the binding position of the first probe 102.

It is necessary for success of the invention that first probe 102 and second probe 105 bind in near proximity, to accomplish the fluorescence necessary by interaction between the fluorescent substances carried by the probes. So, a further constraint is that the two probes are probes within complementary fluorescent properties with emission and excitation wavelength overlap such that one acts as a donor and one an acceptor fluorophore.

As shown in FIG. 1B, nucleic acid target 101 is contacted with a donor probe 102 which binds by complementary sequence with a specific portion of the target. Probe 105 is shown in FIG. 1B as unbound, and as being bound to a quencher probe 106. An important purpose of the quencher probe in one embodiment of the invention is to enhance signal-to-noise ratio in the solution. A dark state is accomplished in this irrespective of acceptor probe concentration.

As further depicted in FIG. 1B the acceptor probe 105, when unbound, has some nucleotide analog subunits occupied by quencher probe 106. Quencher probe 106, in this embodiment, is constructed of a suitable number of nucleic acid or nucleic acid analog subunits to provide for binding through complementary nucleic acid base hybridization in a testing solution and a chemical ligand capable of absorbing energy when adjacent to the acceptor probe 105 fluorescent substance.

The design of the probe sequence for each probe is such that, when two or more suitable probes are bound, the fluorescent substances on each probe are in close proximity to one another, and the combination acts for the transfer of nonradiative energy from the donor to the receptor probe.

FIG. 1C illustrates target sequence 101 with probes 102 and 105 bound in close proximity to the target nucleotide sequence. The quencher probe 106 is designed in a manner that it remains bound in free solution, as depicted in FIG. 1B, but disassociates when acceptor probe 105 binds to the nucleic acid target sequence, and then no longer quenches the acceptor probe 105 fluorescent substance.

When bound to target sequence 101 and aligned properly in close proximity to one another, fluorescent probe 102 is excited by external excitation light 103, typically laser-produced, with a suitable wavelength and intensity to induce emission of nonradiative energy 104 from donor probe 102 to the acceptor probe 105, through Forster resonance energy transfer (FRET). FRET is described in reference T. Forster, Modern Quantum Chemistry, Istanbul Lectures, Part III, 93-137, 1965, Academic Press, New York which is incorporated by reference.

The fluorescent material bound to probe 105, as a result of the absorption of energy 104, radiates photons 107, in a wavelength detectable by a light detection device 108, and the intensity of the light emitted by the fluorescent material of probes 105 is a measure of the density of the target material in the sample.

The above description is based on the circumstance that the probes bind to the target and the near field energy transfer and light emission from probe 105 actually takes place. If it does not, then the target is not the pathogen for which the probes were designed.

The energy transfer between the two probes is mediated by dipole-dipole interaction. Spectroscopically, when donor probe 102 is excited, its specific emission intensity decreases while acceptor probe 105 specific emission intensity increases, resulting in fluorescence enhancement.

In an alternative embodiment of the invention, the process proceeds without the incorporation and binding of a quencher probe to probe 105. The method described above wherein a quencher probe is bound to probe 105 in solution, has advantages over the alternative method, in that background light energy from the free, unquenched acceptor probe 105 will decrease the signal to noise ratio. However, for some applications the alternative embodiment may be preferred.

In some specific embodiments of the invention, donor probe 102 and/or acceptor probe 105 may be implemented as phosphorodiamidate morpholino oligonucleotides or other suitable nucleoside or nucleotide analog, such as LNA (Locked Nucleic Acids), consisting of 2′-O,4′-C-methylene bicyclonucleoside monomers or combinations of nucleotides and nucleotide analogs. Fluorescent substance pair choices include any fluorescent chemical moiety with suitable properties that include the stability, level of brightness, ability to conjugate to the oligomer, and ability to transfer energy to an adjacent, suitably fluorescent substance with which it is paired. The spacing of the oligonucleotides from one another is important to the invention in order to properly align the fluorescent substances. Furthermore, proper placement of the fluorescent substances contributes to successful transmission of light energy from donor probe to acceptor probe.

Advantages of the present invention include, without limitation, that the kit to practice the invention is modular and portable, and facilitates testing in any environment. The reagents used are sufficiently stable to allow easy storage under extreme conditions of heat and cold. While other testing methods use enzymes or proteins that often are constrained by environmental conditions, the elements of the present invention do not require special storage or use conditions, and the kit is thusly deployable in austere environments.

In all embodiments, the method in specific embodiments of the present invention differs significantly from other methods used to detect nucleic acid sequence, partly because amplification of specific nucleic acid sequence is not required. This differs from prior art methods that use polymerases to amplify signal. In embodiments of the present invention, signal may be amplified through concentration of target nucleic acid by nucleic acid purification methods known in the art.

Other prior art methods include detection of pathogens by enzyme-linked immunosorbent assays (ELISA) that rely on specific antibody binding. The present invention does not rely on antibody binding, but on a high-affinity binding based on complementary sequence hybridization that provides higher affinity binding than antibody binding, greater specificity with regard to pathogen identity, and in many cases, can be used during early infection, where production of host antibodies has not yet occurred in an infected subject. The present invention also takes little time to detect the target nucleic acid sequence because there are no amplification steps. A typical test can require as little as 10 min from the time of sample processing to the time of reading the sample and determining the quantity of target in a sample.

In one broad embodiment, the present invention is a method for detection of nucleic acid of a specific sequence for identification of an organism or a sequence produced by an organism or a synthetic nucleic acid sequence present in a sample. In other embodiments, the invention may relate to diagnostic kits for the detection of at least one specific nucleic acid sequence in a sample containing one or more nucleic acids at least one of which nucleic acid is suspected of containing said sequence, which kit comprises, in packaged form, a multi-container unit having (a) one container for oligonucleotide probes for one strand of each different sequence to be detected, which probes are substantially complementary to each strand of each specific nucleic acid sequence such that hybridization to said specific nucleic acid sequence brings probes with fluorescent substances capable of Forster resonance energy transfer into proximity such that energy transfer can be initiated by contacting donor probe with nonradiative energy; (b) A container containing a probe capable of detecting the presence of said hybrids through the Forster resonance energy transfer signature generated from donor and acceptor probe proximity; (c) A means for detecting and quantifying nonradiative energy emitted following Forster resonance energy transfer.

As the sequences of common pathogens are quite well-known, it is within the scope of a skilled artisan to prepare probes for individual ones of such pathogens, and it is to be understood that detection and quantification of pathogens by this unique method is but a single example of use of the invention in different embodiments. In theory, this unique method may be used to detect and quantify any nucleotide from any biologic entity.

As an example of probe design, the following is for Ebola Virus:

### Ebola Virus:

Pair 1 downstream
SEQ ID NO 1
GTCTGTTATGTTCTTGGTCCAATCA Pair 1 upstream,
SEQ ID NO 2
TGGTTCGATACAGCAGTCCGGTCCC - must be capped
on the 3′ end and have a 5′primary amine.

Probes may be designed for detection in this method for a great many other known biologic materials, including pathogens such as Ebola, Zika, HIV, HCV, Salmonella, Tuberculosis, and many others.

As an additional example of the fact of possession of the invention by the present inventors, the following indicates probe sequences and complementary binding for the Zika virus:

| Flaviviru | Accession | Probe Sequence | Predicted | |
|---|---|---|---|---|
| Target | Number | (5′-3′) | Temp | |
| Zika | KX062045.1 | SEQ ID NO 3 | 100.5° C. | SEQ ID NO 5 |
| Virus | | TGTGCGTCCTTGAACT | | GAAG[CACTGGTAGAGTTCAAGGACG |
| | | CTACCAGTG | | CACA]TG[CCAAAAGGCAAACTGTCGT |
| | | SEQ ID NO 4 | 94.5° C. | GTTCT]AGGGAGTCAAGAAGGAGCA |
| | | AGAACCACGACAGTTT | | GTTCACACGGCCCTTGCTGGAGCTC |
| | | GCCTTTTGG | | TGGAGGCTGAGATGGATG |

Similar information may be provided for many other circumstances as indicated above.

It is important to note, because the invention is intended to provide a portable system usable in difficult field conditions, the temperature range is very broad with a range of 32 degrees F. to 140 degrees F.

Further, while most molecular biology assays use standard buffers such as Trisma buffer with EDTA with a pH of 7.0 to 8.5, such buffers are optimized for DNA binding. The probes in the embodiments of the present invention are phosphorodiamidate Morpholino oligomers (PMOs). One of the unique things about the PMOs is that they do not require ionic buffers the same way that normal DNA does.

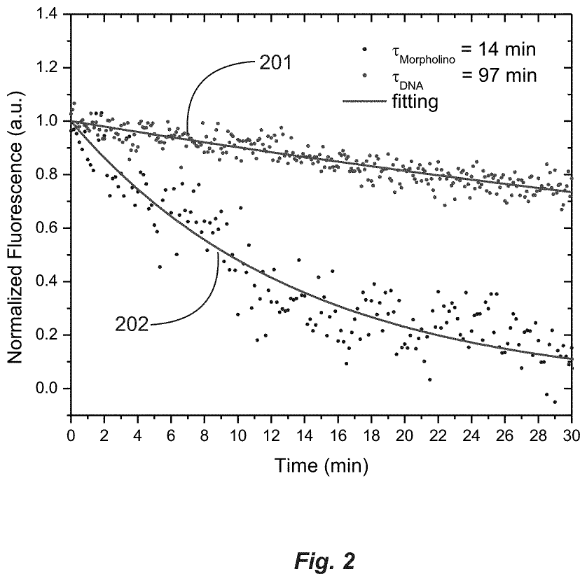

Time of annealing is important to the detection scheme. FIG. 2 is a graph illustrating a time course 201 of 1 nM Donor DNA Probe compared to a time course 202 for a Morpholino Probe binding to 1 nM target DNA. The binding curve with PMO is much faster over time. This means that there may be more confidence in our measurements in a short time frame.

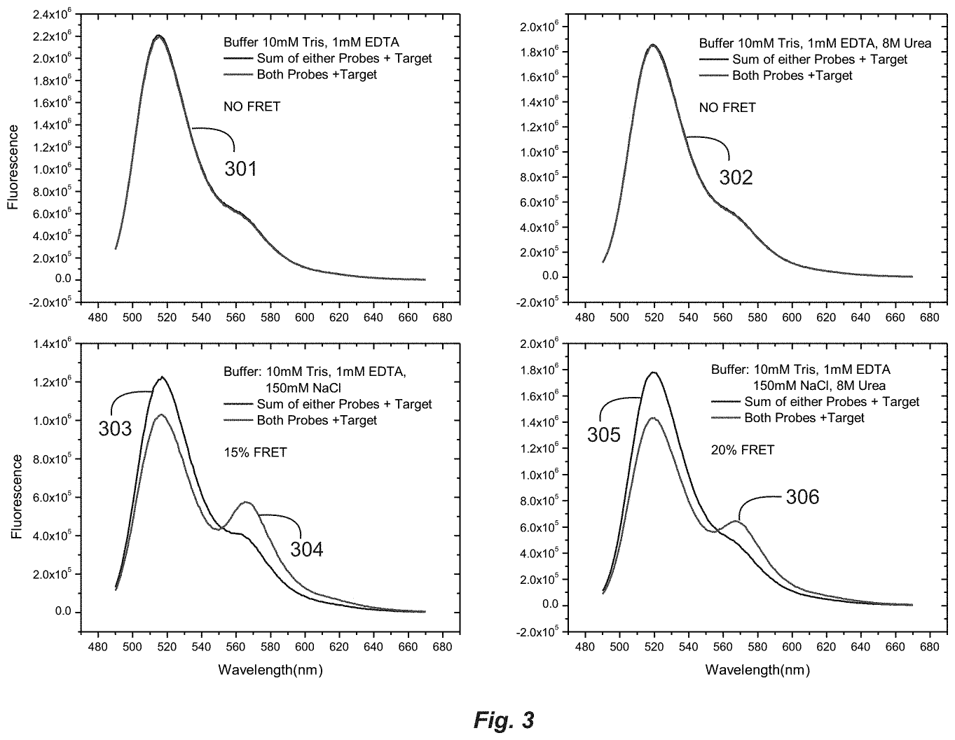

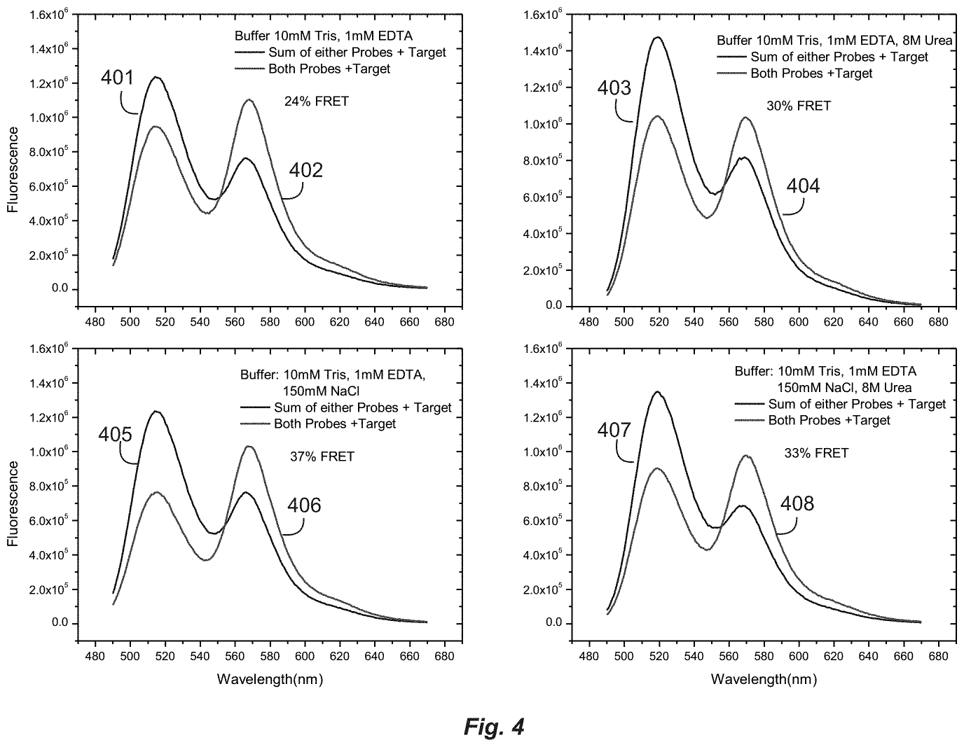

FIG. 3, which illustrates DNA probes labeled with Alexa488 and Alexa 546 bound to target DNA, shows a lack of FRET when the assay is conducted at low ionic strength (upper left). Curve 301 at upper left is a superposition of curves for both a sum of either probe plus target, and sum of both probes plus target. The superposition of the curves indicates no FRET takes place. Curve 302, at upper right shows fluorescence for either probe plus target, and sum of both probes plus target, at low ionic strength under denaturing conditions. Curves 303, for sum of either probe plus target, and 304 for sum of both probes plus target, shown at lower left, are for condition where 150 mM NaCl is added. The assay works under this condition. It also works under denaturing conditions, as shown by curves 305 for sum of either probe plus target, and 306 for sum of both probes plus target, at lower right, but is somewhat diminished. Compare that to FIG. 4, depicting Morpholino probes labeled with FITC and Alexa546 bound to target DNA, where PMOs are used, and it is seen that there is significant signal in all conditions.

In FIG. 4, upper left, curve 401 is for sum of either probe plus target, and curve 402 is for sum of both probes plus target, with buffer 10 mM, 1 mM EDTA(hydroxymethyl)aminomethane (Tris), and 1 mM Ethylenediaminetetraacetic acid (EDTA). This shows 24% FRET. In FIG. 4, upper right, curve 403 is for sum of either probe plus target, and curve 404 is for sum of both probes plus target, with buffer 10 Mm TRIS, 1 mM EDTA, and 8M Urea. This shows 30% FRET. In FIG. 4, lower left, curve 405 is for sum of either probe plus target, and curve 406 is for sum of both probes plus target, with buffer 10 mM TRIS, 1 mM EDTA, and 150 mM sodium chloride, NaCl.

In FIG. 4, lower right, curve 407 is for sum of either probe plus target, and curve 408 is for sum of both probes plus target, with buffer 10 mM TRIS, 1 mM EDTA, 150 mM NaCl, and 8M Urea. Taken all together this data indicates the assay in various embodiments of the present invention would be robust under conditions that would not allow FRET with DNA.

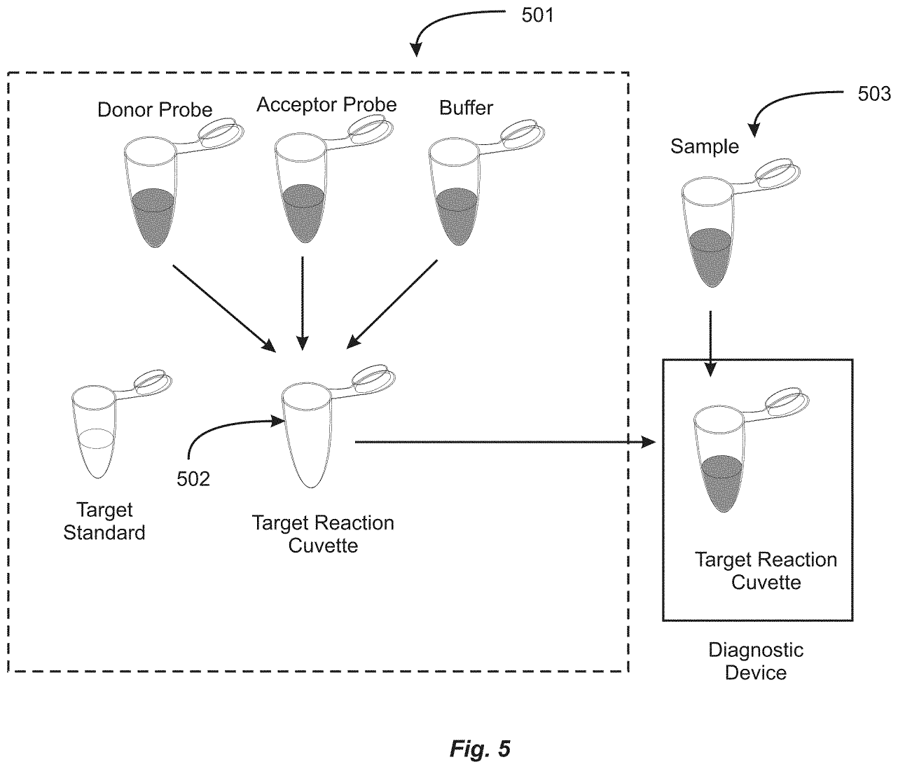

FIG. 5 illustrates a generalized kit 501 in an embodiment of the invention, wherein the donor probe, the acceptor probe and the buffer are stored in separate cuvettes, which may be carried in a convenient container. In practice of the invention the donor, acceptor and buffer are mixed in a target reaction cuvette 502. In different circumstances different donors, acceptors and buffers may be mixed, depending on a target nucleotide sought, or expected to be present in a sample. Depending on expected target, one or another target reaction cuvette 502 is accessed, and a sample 503 is added as shown to the right of kit 501. The target reaction cuvette is specific to a certain diagnostic device, such as a spectrophotometer, which can be used to detect fluorescence and quantitative information.

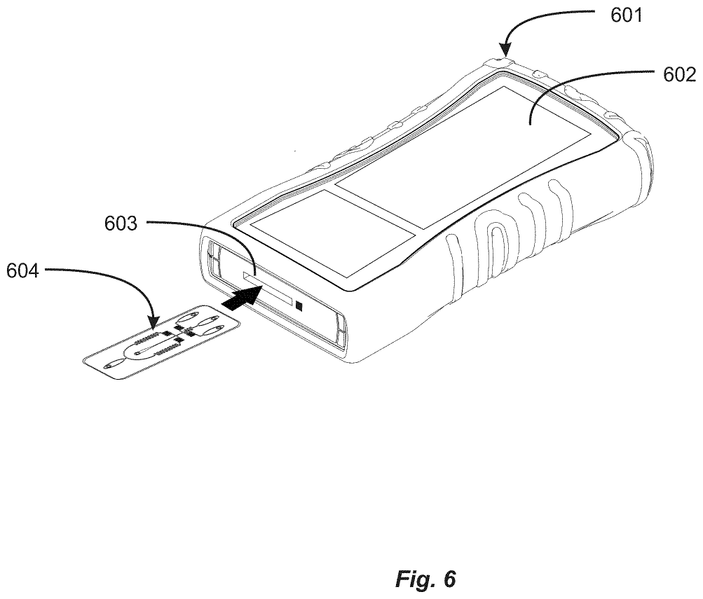

FIG. 6 illustrates a diagnostic device 601 in another embodiment of the invention, having a display 602, power and I/O apparatus not detailed, and an input slot 603 for accepting a diagnostic assay cartridge 604. Assay cartridges may be prepared as needed and inserted in the diagnostic device, wherein laser light is induced to activate the donor probe, and the activity, if any, of the acceptor probe may be detected and measured. Activity and quantitative information is displayed on display 602.

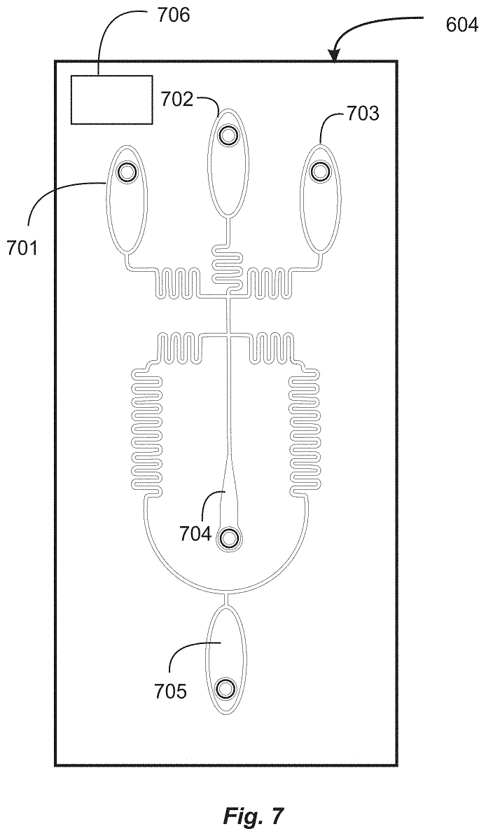

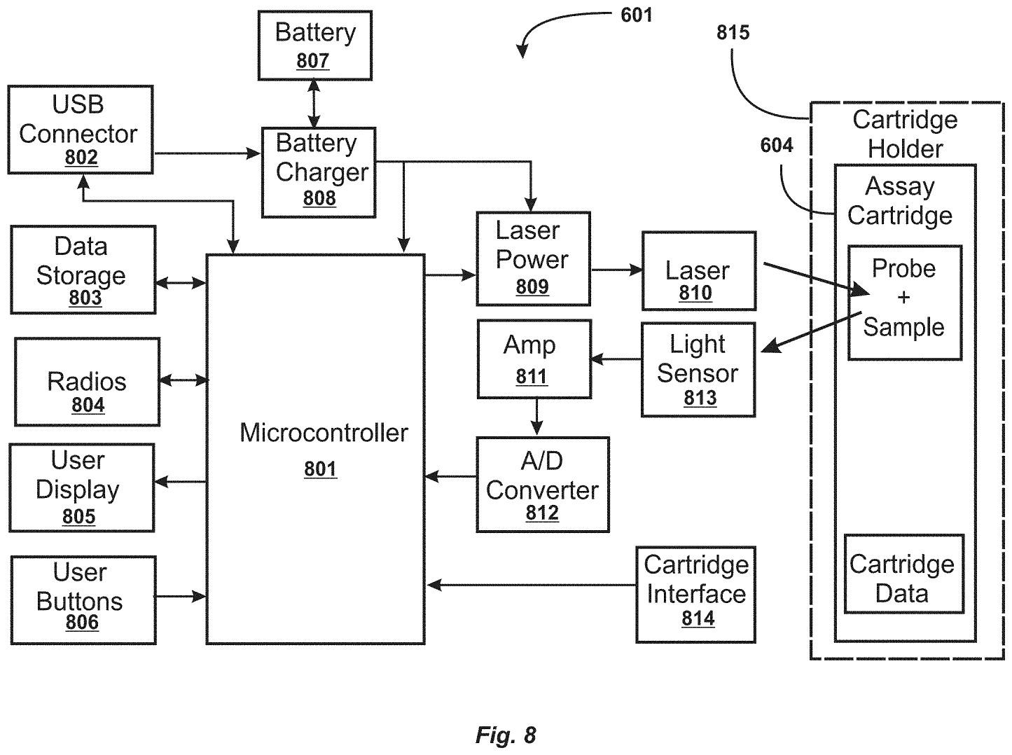

FIG. 7 is a plan view of one diagnostic assay cartridge 604, illustrating a donor probe chamber 701, an acceptor probe chamber 702, a buffer chamber 703, a target reaction chamber 704 and a sample chamber 705, as well as interconnection channels. Each of the chambers has an interface by which probes, buffer or sample may be added. In embodiments of the invention similar, preloaded assay cartridges are provided as target standards, which may be entered into the diagnostic device for comparison and calibration. Element 706 is a machine-readable element or indicia wherein information regarding the cartridge is stored, to be read by apparatus in the hand-held computerized device. This element may be a bar code, QR code, or a chip with electronic information accessible by apparatus and circuitry in the hand-held device. Access to such information is described further below, with reference to FIG. 8.

FIG. 8 is a block diagram of the elements of diagnostic device 601 in an embodiment of the invention. Different elements indicated as blocks in FIG. 8 may be as simple as a single chip, or a small number of chips working in concert to provide a function. Linear connections shown as arrows between elements are meant to illustrate power and communication, although not shown separately.

Diagnostic device 601 is provided with a USB port 802 and circuitry in the device which may be used to connect to very commonly available USB chargers to run the system and to charge the battery. The USB sub-system may also be used to update system software stored in the Microcontroller or other development tasks when necessary.

When not connected to a USB charger, the system can run from the energy stored in battery 807. The system draws low power. A laser, described further below, is the largest energy usage element in the system, but is only active for a very short period of time when running an assay. The handheld unit may accommodate a battery large enough to run a large number of assays on battery power between charges.

Functions of the diagnostic device are controlled through an inexpensive, low powered microcontroller 801. A CPU in the microcontroller does not require high performance for controlling the Diagnostic System, thus it can be inexpensive and low powered. Microcontrollers that are commonly available have built-in RAM and Flash for program execution, reducing the complexity of the system. Various GPIO (general purpose input/output) pins, and simple serial interfaces, such as I2C and SPI, are commonly available on microcontrollers. These pins may provide interfaces to other elements of the device.

A data repository 803 is provided, connected to the microcontroller, and may be a single Flash memory chip. The memory is provided to hold assay results when the system is in use.

The system has one or more radios 804. In one embodiment, a Bluetooth Low-Energy (BLE) radio may connect to a nearby smartphone. An application in the smartphone may be provided to receive assay results as same are created, and data on assays that have been stored in the Diagnostic System's Data Storage. The smartphone application may display results on the phone display, and when a network connection is available on the smartphone's Wi-Fi or cellular radios, the smartphone app may forward the assay data to a centralized database for storage and further analysis. The diagnostic device might further incorporate Wi-Fi or cellular radios for directly connecting to networks when same are available, although this increases hardware cost and uses more energy from the battery. Thus, BLE is preferred. in one embodiment, when the radio of the diagnostic device makes contact to the network, the data in the memory is transferred, and the storage memory is cleared for future assay results.

User display 805 and user buttons 806 are used to interact with a person setting up the system or running assays. During setup, for example, the display and buttons might be used to configure the system to connect to a particular phone via BLE, or to a particular Wi-Fi network. During operation, the display and buttons might be used to:
Display battery energy remaining, and charging status, so the operator knows when charging is required.
Show cartridge identification data so an operator can verify the correct type of cartridge is present.
Start a assay and then display the results for immediate use by the care giver.
When no network is present, the operator can be informed how many assay results are currently stored, and how many more results can be stored in the Data Storage before it is full.
Display the status of the network/phone radio connection. The operator can then take action if a network connection needs to be established.

Laser power circuitry 809 controls power sent to a laser 810 under software control from the Microcontroller. Due to relatively large power requirements of the laser, it is only powered when needed for a short time to run an assay. Laser 810 is used to illuminate the mixture of probe+sample to activate FRET during an active assay. Light sensor 813 is a very sensitive device that converts incoming light, generated by FRET in the Probe+Sample, into an analog voltage. Due to relatively low light energy generated by a small sample, the analog voltage coming from the light sensor must be amplified to enable detection of weak signals that may be generated by low concentrations of targets nucleotide in the sample. Thus, an amplifier circuit 811 may be a high performance, low drift, low offset voltage, high gain circuit to provide reliable and useful signal to an A/D converter 812.

Cartridge holder 815 is a slot in device 601 that matches the size and shape of assay cartridge 604. The holder positions the Cartridge such that the probe+sample is properly aligned with the laser and light sensor. It also holds a cartridge data block in correct position relative to the cartridge interface. The holder may have a door that closes after insertion of the cartridge, or is tightly fitted to the Cartridge such that external light cannot enter the probe+sample area and affect any result of the assay. The door may have an eject mechanism the operator can use to open the door and eject the cartridge, or the cartridge can be built to extend out of the tight fitting slot so the operator can grasp it to remove it.

Assay cartridge 604 in some embodiments has a data store (see element 706 in FIG. 7) incorporated that records data that may be determined during manufacture. This data might include some or all the following:
Unique serial number, for tracking
Assay name, e.g. “Ebola”, for presenting to the operator to ensure the correct cartridge is being used.
Date of manufacture and Lot number, for tracking
A data table for converting a sensor reading to virus load. Note this table could be provided from servers over the network when available, perhaps with refined information created since the Cartridge was manufactured. Another option is to send the Cartridge Data and A/D converter output over the network to servers that, along with the Cartridge Data, is used to arrive at a assay result which is sent back to the device for display to the operator.

Cartridge interface technology might use near field communications (NFC) radio, or optical technology, such as a QR code and camera, or contacts similar to those found on an SD Card, or even the technology of “chip and pin” credit cards. A preference is for technology that is robust and low cost. NFC seems to be a good match, as it doesn't rely on clean contacts nor clean optics in order to work.

The diagnostic device computing core runs embedded software to both control the device and to communicate with the rest of the diagnostic overall system. The embedded code provides operating instructions to read data from an inserted diagnostic cartridge and to appropriately power the laser and optical detector, take accurate readings of FRET results from a sample reaction with the reagents, compare the FRET results against a target data fingerprint stored within the diagnostic cartridge, to display results to the user via the onboard display, securely store the data for later use, and to communicate the information wirelessly to a mobile application for advanced analysis. Base functionality, like pairing with mobile devices and Wi-Fi, follow well known steps as known in computing arts, and are not further described herein.

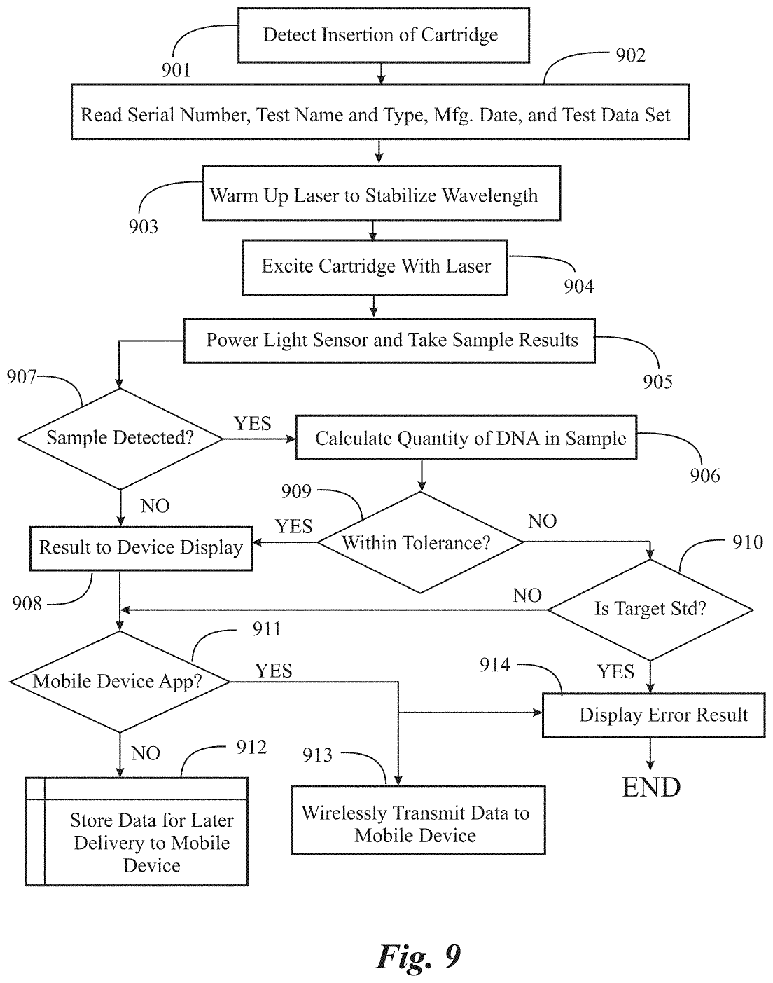

FIG. 9 is a flow chart illustrating a process cycle in one embodiment of the invention for either a target standard cartridge, or for a diagnostic target cartridge.

Following the example depicted by FIG. 9, at step 901 a user inserts an assay cartridge into the cartridge holder of device 601. The device senses the insertion, and at step 902 reads the embedded data associated with the device, including, for example, a serial number, a test name and type, a manufacturing date, and a test data set. At step 903 the device initiates power to laser 810 (FIG. 8), and provides a warmup period to stabilize the wavelength. At step 904 the laser is exposed to excite the cartridge. At step 905 light sensor 813 is powered on and used to take sample results. At step 907 it is determined whether or not a sample has been detected.

If at step 907 a sample is detected, the quantity of DNA in the sample is calculated at step 906. If not, the lack of a sample is communicated to the device display at step 908. If, at step 906 a quantity of DNA has been determined, at step 909 it is determined whether or not the quantity is within a specified tolerance. If so, that result is communicated to the display at step 908. If the calculation was outside of tolerance at step 909. At step 910 it is determined whether or not the target is a standard target. If so, the error is displayed at 914, and the process is finished.

If at step 910 it is determined the target is not a standard target, then the device looks for a mobile device app at step 911, and if found, wirelessly transmits the determined data from the assay to the mobile device app at step 913, and the process ands. If, at step 911 no mobile device app is found, the determined data for the assay is stored in data store 803 for later delivery to a mobile device, or other data collection apparatus.

The skilled person will understand that the process illustrated and described with reference to FIG. 9 is exemplary, and that the process may take alternative forms and steps.

In an embodiment of the invention a molecular diagnostic database is generated and maintained. This database has multiple components. A first component is a set of diagnostic target signatures, which is a collection of quantitative laboratory test data generated by controlled analysis of each target DNA sequence that a diagnostic probe kit has been design to detect. This data set supplies the target signatures that will be used to identify and calculate quantity of found target DNA added to a diagnostic assay cartridge and processed through the diagnostic handheld device.

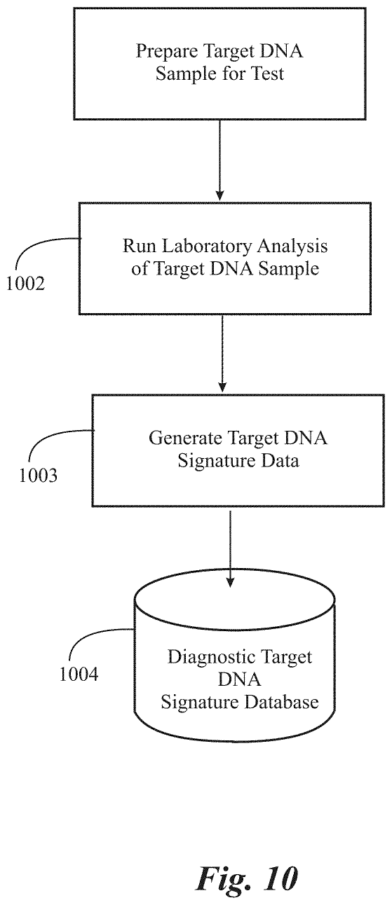

FIG. 10 illustrates a simple, serial process that is performed for each nucleotide that is expected to be a target for analysis by the system of the invention. At step 1001 a target sample DNA is prepared for test. At step 1002 a laboratory analysis is made of the target sample. At step 1003 DNA signature data is generated for the subject target, and at step 1004 the result for that subject target DNA is recorded in a diagnostic target signature database.

A second database component is the distributed target DNA signature data set that is saved onto each diagnostic assay cartridge for the particular target DNA test that the cartridge is meant to test for. The data on the Diagnostic cartridge in one embodiment is:
serial number
Target DNA test identification for target the cartridge can test for.
Date of manufacture
Target DNA signature data set used in calculating if target is found and quantity of DNA present in sample.

A third database component is generated by the diagnostic device and the mobile diagnostic application. Below is the data that each contributes to the overall data set:
Diagnostic Device:
   Time of test
   Calibration result data performed on target standard prior to test
   Test result derived from sample under test
   Time of delivery of test data sent to diagnostic service application
Mobile Diagnostic Application:
   Known recorded geo-location in latitude/longitude pairs of diagnostic device location when target sample is taken
   Additional information used for security and HIPPA compliancy

A forth database component comprises are queries developed to produce advanced digital epidemiology results when processed against data collected from the previous three database components. Data stored in the real time database will be the basis for advanced analysis of DNA, allowing users an ability to track DNA location and movement in real time as target samples are processed by diagnostic devices in use around the world. These software tools will allow local, regional and global views of molecular diagnostics and epidemiology in real time not before seen. This will be very disruptive in the molecular diagnostics industry truly allowing early detection and early response to the DNA data collected.

Software operable in the mobile device, providing the mobile application discussed above in several instances, allows a healthcare worker an ability to take Point of Care samples via an easy one touch process, and to access the molecular diagnostic data from anywhere on a diagnostic cloud network. The mobile application in some embodiments is a secure application communicating to the diagnostic device via standard wireless protocols. The mobile diagnostic application has two primary functions in one embodiment. First it acts as a wireless remote link to the diagnostic device giving a full, easy to use user interface with more features and capabilities than the diagnostic devices limited capabilities. Secondly, the mobile software gives a user ability to remotely send and receive data, queries and advanced analysis to the Real Time Diagnostic Epidemiology Database wirelessly via the cloud. This wireless capability of the software gives the entire system the ability to track epidemiology anywhere in real time.

While the foregoing written description of the invention enables one of ordinary skill to practice the invention, the skilled artisan will understand and appreciate the existence of variations, combinations, and equivalents of the specific embodiments described. The invention is therefore limited only by the language of the claims below.

1. A method for detecting and quantifying a target nucleic acid sequence in a sample, comprising the steps of:
(a) combining in a single solution:
i. a first phosphorodiamidate morpholino oligomer (PMO) probe comprising a first fluorescent dye actionable under exposure to incident light to act as a donor, the first probe having a sequence complementary to a first portion of the of the target nucleic acid sequence;
ii. a second PMO probe comprising a second fluorescent dye actionable as a receptor, the second probe having a sequence complementary to a second portion of the target nucleic acid sequence, the second portion in near proximity to the first portion; and
iii. a sample material suspected of comprising strands of the target nucleic acid sequence;
(b) exposing the combined solution to incident light at a specific wavelength known to cause the first fluorescent dye of the first PMO probe to donate energy in a non-radiative manner to the second fluorescent dye of the second PMO probe, causing the second fluorescent dye to fluoresce as a result; and
(c), detecting and measuring fluorescence of the second fluorescent dye by a light detection device, detection indicating presence of the target nucleic acid sequence in the sample.

2. The method of claim 1 wherein the first and second PMO probes are added together or sequentially to a solution containing the sample material suspected of comprising strands of the target nucleic acid sequence.

3. The method of claim 1 wherein the sample material suspected of comprising strands of the target nucleic acid sequence is added to a solution containing the first and second PMO probes

4. The method of claim 1 wherein the light detection device is enabled to record intensity of the emitted photons as an indication of the quantity of the target nucleic acid present.

5. The method of claim 1 further comprising adding a quencher dye having substantially lower binding affinity than the target nucleic acid, which quencher dye binds to the acceptor dye in solution, absorbing energy from the acceptor dye when the second PMO probe is free in solution, and disassociates when and if the second PMO probe binds to the target nucleotide.

6. The method of claim 1 wherein the first and second PMO probes contain multiple donor or acceptor dyes, enabling multiplexed amplification of signal from more than two oligomers.

7. The method of claim 1 wherein the first and second PMO probes are prepared complementary to one of the nucleic acid sequences Ebola virus, Zika virus, tuberculosis virus, salmonella virus, HIV virus, or HCV virus.

8. The method of claim 1 further comprising a step for comparing result of measurement of light emission of the second fluorescent dye to prepared standards, to determine quantity of target nucleic acid sequence in the sample.

9. A kit for detecting and quantifying a target nucleic acid sequence in a test sample, comprising:
a first container holding a sample solution possibly comprising the target nucleic acid;
a second container holding, in solution, a first phosphorodiamidate morpholino oligomer (PMO) probe comprising a first fluorescent dye actionable under exposure to incident light to act as a donor, the first PMO probe having a sequence complementary to a first portion of the of the target nucleic acid sequence;
a third container holding, in solution, a second PMO probe comprising a second fluorescent dye actionable as a receptor, the second probe having a sequence complementary to a second portion of the target nucleic acid sequence, the second portion in near proximity to the first portion;
a light source enabled to illuminate contents of the first container at a wavelength inducing the first fluorescent dye to donate energy in a non-radiative manner to the second fluorescent dye; and
a device to collect and measure fluorescent light emissions from the acceptor probe dye.

10. The kit of claim 9 further comprising a fourth container holding, in solution, a quencher dye having substantially lower binding affinity than the target nucleic acid, which binds to the acceptor dye in solution, absorbing energy from the acceptor dye when the second oligomer probe is free in solution, and disassociates when and if the second oligomer probe binds to the target nucleotide.

11. The kit of claim 9 wherein the light detection device is enabled to record intensity of the emitted photons as an indication of the quantity of the target nucleic acid present.

12. The method of claim 9 wherein the first and second PMO probes contain multiple donor or acceptor dyes, enabling multiplexed amplification of signal from more than two probes.

13. The kit of claim 9 wherein the first and second PMO probes in the second and third containers, respectively, are implemented to be complementary to one of nucleic acid sequence of Ebola virus,

14. The kit of claim 9 wherein the first and second oligomer probes in the second and third containers, respectively, are prepared to be complementary to one of the nucleic acid sequences Ebola virus, Zika virus, tuberculosis virus, salmonella virus, HIV virus, or HCV virus.

15. A portable detection system for detecting and quantifying a target nucleic acid sequence in a sample, comprising:
a hand-held, computerized appliance having a cartridge holder for holding a sample cartridge, a laser for illuminating a sample in a cartridge in the cartridge holder, a light sensor for detecting light emission from the sample in the cartridge, and wireless circuitry for communication with a network-connected communication device; and
a cartridge configured to insert and withdraw from the cartridge holder, the cartridge comprising a donor probe chamber, an acceptor probe chamber, a sample chamber, and a target reaction chamber, with interconnecting internal channels;
wherein a cartridge implemented with donor and acceptor probes specific to a particular target nucleotide in respective chambers is loaded with a sample in the sample chamber, and inserted into the cartridge holder, where the sample and probes are mixed in the reaction chamber and illuminated by the laser, and intensity determined by the light detector is communicated wirelessly to the network-connected communication device.

16. The system of claim 15 further comprising an application executing on the network-connected communication device, enabling digital storage of results of assays and communication with a remote, network-connected database.

17. The system of claim 15 further comprising the cartridge having machine readable information thereon, identifying at least a unique serial number for tracking, and further comprising apparatus in the hand-held, computerized appliance, enabling reading of the machine-readable indicia.

18. The system of claim 17 wherein the machine-readable information is in the nature of a bar code or Quick Response (QR) code, and the reading apparatus is optical in nature.

19. The system of claim 17 wherein the machine-readable indicia is electronic in nature, and the reading apparatus comprises wireless circuitry for accessing the data on the cartridge.

20. The system of claim 19 wherein the reading apparatus comprises electronic contacts between the cartridge and circuitry in the hand-held device.

21. The system of claim 17, wherein, in addition to a unique serial number, the information comprises one or more of an assay name, date of manufacture and lot number, and one or more data tables.


SEQUENCE LISTING (5 sequences)

SEQ ID NO: 1 - DNA, 25 bp, Ebola virus
gtctgttatgttcttggtccaatca

SEQ ID NO: 2 - DNA, 25 bp, Ebola virus
tggttcgatacagcagtccggtccc

SEQ ID NO: 3 - DNA, 25 bp, Zika Virus
tgtgcgtccttgaactctaccagtg

SEQ ID NO: 4 - DNA, 25 bp, Zika Virus
agaaccacgacagtttgccttttgg

SEQ ID NO: 5 - DNA, 118 bp, Zika Virus
gaagcactggtagagttcaaggacgcacatgccaaaaggcaaactgtcgtggttctagggagtcaagaaggagcagttcacacggcccttgctggagctctggaggctgagatggatg